(19) United States
(12) Patent Application Publication
(10) Pub. No.: US 2013/0129565 A1
(43) Pub. Date: May 23, 2013

(54) FILTER AND DEVICE FOR TREATING AIR

(71) Applicant: Ah Eng Siaw, Singapore (SG)

(72) Inventor: Ah Eng Siaw, Singapore (SG)

(21) Appl. No.: 13/813,763

(22) PCT Filed: Aug. 3, 2010
(86) PCT No.: PCT/MY10/00137

§ 371 Date: Feb. 1, 2013

(51) Int. Cl. A61L 9/20

(52) U.S. Cl. CPC A61L 9/205; B82Y 99/00
USPC 422/4; 422/120; 422/121; 977/777

(57) ABSTRACT

A filter (100) comprising at least one antibacterial layer (101) consisting of nano-silver particles and at least one ultraviolet photocatalytic layer (102).

### FIELD OF INVENTION

This present invention relates to a filter and device for treating air. In more particular, the present invention relates to a photocatalytic filter and device for treating air.

### BACKGROUND OF THE INVENTION

An emerging air treatment technology is based on a photocatalytic oxidation that converts fine particles and toxic gases into safer compounds. Basically, a photocatalytic air treatment shall include the use of broad spectrum ultraviolet light which reacts with a photocatalyst to create hydroxyl radicals and super-oxide ions for oxidizing volatile organic compounds and eliminating microorganisms adsorbed on the catalyst surface.

One common photocatalyst being used for an air purifier is titanium dioxide. By bombarding the titanium dioxide with lights of certain wavelengths, electrons in the material's valence band are excited into the conduction band. As a result, the electrons are free to move and their energy can be utilized to split up nearby water and oxygen molecules into hydroxyl radicals and super-oxide ions.

Hydroxyl radicals are among the most powerful oxidizers and stronger than chlorine, ozone and peroxide but consequently the radicals are very short lived. The oxidizers can break the bonds of organic substances such as germs and volatile organic molecules into smaller compounds until only carbon dioxide and water vapor are left.

There is some prior arts relating to several photocatalytic filters which can be installed in a device for treating air.

A Japanese Patent No. 2006017360 describes an air filter of a forming pleat which sticks to at least one of dust, an odor molecule, a virus and bacilli presented in the air. In the prior art, it also discloses an air conditioner provided with an air purifying unit containing the air filter.

Another Japanese Patent No. 2006280428 discloses an adsorption filter layer, provided in an inspired air flow path, has breath-ability and a light blocking effect to adsorb harmful gas component. Furthermore, the prior art claims of using a disinfection odor removal filter provided with a photocatalyst filter layer which has breath-ability being laminated by exhaust side of the adsorption filter layer to decompose an odor component in response to ultraviolet radiation from an exhaust side.

A Japanese Patent No. 2009028473 describes a filter which is coated with nano silver installed together with a germicidal lamp into a wind-speed control type ultraviolet irradiation air sterilizer. Based on the prior art, the air sterilizer filters microorganisms such as bacteria, virus and true fungi which float in the air of a room or in a car.

One improvement on photocatalytic oxidation technology includes addition of other elements with titanium dioxide as the catalyst. Some examples of the elements are vanadium, copper, zinc, rhodium, silver and nickel.

Great attention has been focused on using nano-silver particles in a filter for treating air. It has been examined that silver can provide natural anti-bacterial, anti-viral and anti-fungal benefits. When the nano-silver particles come into contact with a bacteria or virus, they suppress the cell's nutrient transport, attack the cell membrane and interfere with cell division to hinder the reproduction of the germs.

It is desirable to invent a filter comprising nano-silver particles which can be easily installed into a device for treating air effectively via removing a variety of contaminants.

### SUMMARY OF THE INVENTION

The primary object of the present invention is to invent a filter comprising nano-silver particles or a combination with other photocatalytic elements for treating air effectively via removing a variety of contaminants.

Another object of the present invention is to invent a device installed with a filter comprising nano-silver particles or a combination with other photocatalytic elements for treating air effectively via removing a variety of contaminants.

At least one of the preceding objects is met, in whole or in part, by the present invention, in which the embodiment of the present invention describes a filter (100) comprising at least one antibacterial layer (101) consisting of nano-silver particles and at least one ultraviolet photocatalytic layer (102).

In the present invention, the filter comprises of at least one antibacterial layer and at least one ultraviolet photocatalytic layer while further comprising at least one electrostatic layer. The antibacterial layer contains nano-silver particles which are effective in removing microorganisms and odors.

### BRIEF DESCRIPTION OF THE DRAWINGS

FIG. 1 shows a schematic view on the cross-section of several preferred filters (100) for treating air.

FIG. 2 shows a schematic view of a ceiling suspended unit as a preferred device (106) for treating air.

FIG. 3 shows a schematic view of another ceiling suspended unit as a preferred device (106) for treating air.

FIG. 4 shows a schematic view of a floor standing unit as a preferred device (106) for treating air.

FIG. 5 shows a schematic view of another floor standing unit as a preferred device (106) for treating air.

FIG. 6 shows a schematic view of an air handling unit as a preferred device (106) for treating air.

### DETAILED DESCRIPTION OF THE INVENTION

One skilled in the art will readily appreciate that the present invention is well adapted to carry out the objects and obtain the ends and advantages mentioned, as well as those inherent therein. The embodiment described herein is not intended as limitations on the scope of the invention.

The present invention discloses a filter (100) comprising at least one antibacterial layer (101) consisting of nano-silver particles and at least one ultraviolet photocatalytic layer (102). The filter (100) shall further comprise at least one electrostatic layer (103).

In the present invention, the antibacterial layer (101) has a loading of nano-silver particles ranging from 0.1% to 5% by weight. The nano-silver particles are ranging from 10 nm to 25 nm in size. It is important that silver particles are utilized in nano-sized range to provide bigger surface area as compared to common micro-sized silver particles.

Bigger surface area can provide a larger contact surface which eliminates microorganisms more effectively. In an example, 1 gram of 20 nm silver particles equals to surface area of 50 m².

The nano-silver particles which come in contact with the bacterium and fungus can adversely affect the cellular metabolism and inhibit the cell growth such as suppression on respiration, basal metabolism of electron transfer system and transport of substrate in the microbial cell membrane. As a result, the nano-silver particles are able to inhibit multiplication and growth of those bacteria and fungi which may cause infection, odor, itchiness and sores.

In addition, the antibacterial layer (101) consisting the nano-silver particles can further include other elements such as zinc oxide, calcium oxide, magneisum oxide, silica or any combination thereof. The antibacterial layer (100) can destroy microorganisms such as various kinds of viruses and bacteria and also oxidize odor from the air.

As claimed in the present invention, the ultraviolet photocatalytic layer (102) will consist of titanium oxide which is responsive to an ultraviolet or florescence light source to oxidize virus, bacteria, mold, fungus, odor, volatile organic compounds and toxic gases. However, the electrostatic layer (103) functions to trap dust from the air passing across the filter (100).

In the present invention, the ultraviolet photocatalytic layer (102) has a loading of titanium oxide ranging from 0.1% to 21% by weight.

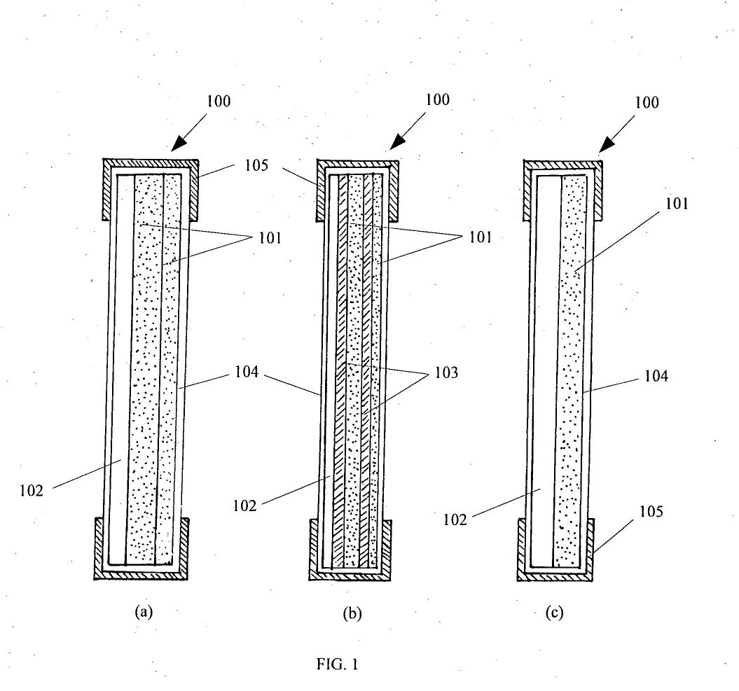

In one preferred embodiment of the present invention, the basic combination of a preferred filter (100) shall comprise one layer of antibacterial layer (101) and one layer of ultraviolet photocatalytic layer (102) wherein the antibacterial layer (101) shall be consisted of nano-silver particles and optionally added with zinc oxide and/or silica. The preferred filter (100) as described herein is shown in FIG. 1(c).

The present invention describes another preferred filter (100) in FIG. 1(a), illustrating an antibacterial layer (101) which is sandwiched between another antibacterial layer (101) and an ultraviolet photocatalytic layer (102).

Based on the present invention, FIG. 1(b) is the most preferred filter (100) and it has a core of antibacterial layer (101), two electrostatic layers (103), one outer antibacterial layer (101) and one ultraviolet photocatalytic layer (102).

Besides, the filter (100) may be protected by a polymeric net (104). Taking for an example, the antibacterial layer (101) and/or ultraviolet photocatalytic layer (102) of the filter (100) can be coated preferably with a polymeric net (104) made of polypropylene, polyester, woven or any combination thereof.

In addition, the filter (100) is also protected by a metal casing (105) preferably made of aluminum, galvanized steel or a combination thereof.

Hereinafter, the present invention discloses a device (106) for treating air comprising at least one ultraviolet light source (107) and at least one filter (100) as described previously positioned to be exposed to the ultraviolet light source (107). The device (106) may further comprise at least a fan (108), a cooling or heating coil (110) or any combination thereof.

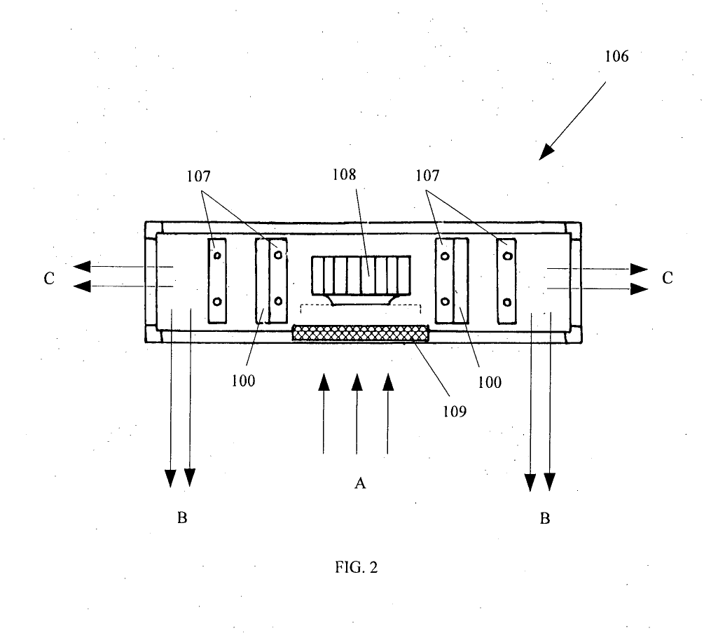

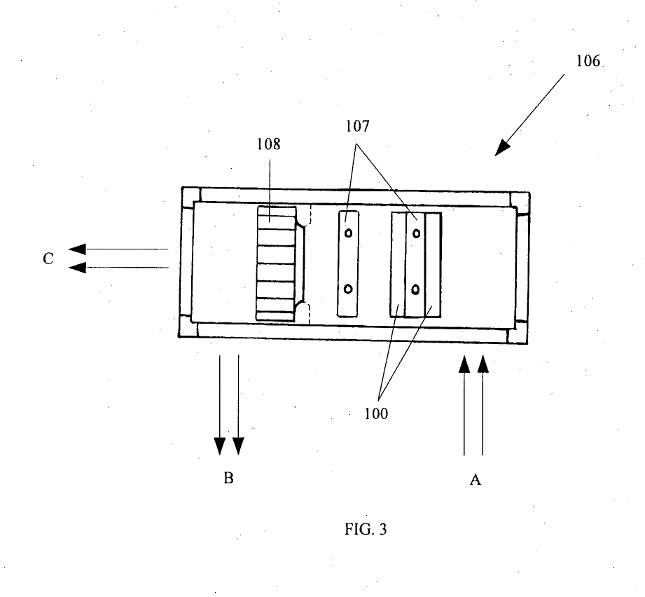

In the present invention, each of the FIG. 2 and FIG. 3 has shown a ceiling suspended unit as a preferred device (106) for treating the air. In FIG. 2, air is withdrawn indicated by the arrows labelled as A into an inlet, wherein an electrostatic filter (109) containing nano-silver particles and/or zinc oxide can be placed at the inlet to capture smoke by electrostatic force.

Optionally, a fan (108) is installed near the inlet to direct the air into the device (106) and pass the air across the filter (100). At least one filter (100) is positioned to be exposed to at least one ultraviolet light source (107) for removing various types of air contaminants including dust, microorganisms and organic volatile compounds.

In another preferred embodiment of the present invention, the filter (100) as installed in the device (106) shall have a photocatalytic layer (102) which contains titanium oxide preferably supported on a wire mesh made of metal such as aluminum. The treated air will be directed out from the device (106) indicated by the arrows labelled as B or C or a combination thereof.

The preferred device (106) of another ceiling suspended unit in FIG. 3 shows that the air is entered as indicated by the arrows starting around point A from an inlet into the device (106). Air passes across at least one filter (100) positioned to be exposed to at least one ultraviolet light source (107) and a fan (108) will blow out the treated air to a point labelled as B or C or a combination thereof.

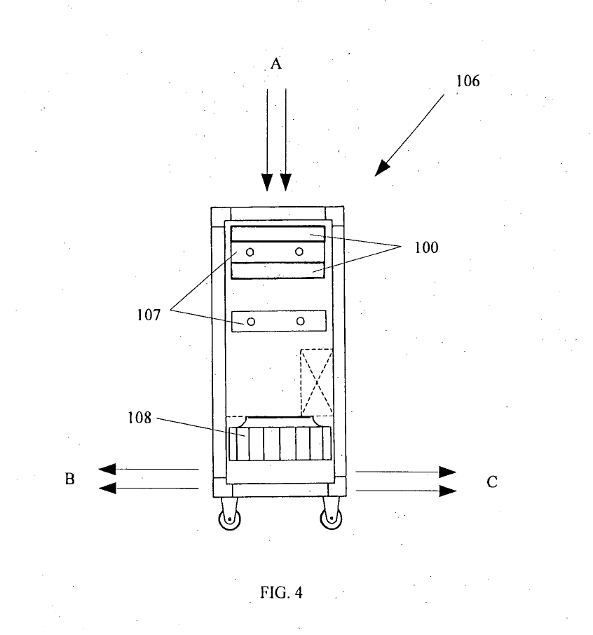

In FIG. 4, a preferred device (106) of a floor standing unit is used for treating air. Air will be withdrawn indicated by arrows labelled as A preferably from a top inlet and into the device (106) where at least one filter (100) is positioned to be exposed to at least one ultraviolet light source (107) to generate hydroxyl radicals for treating the air. The treated air shall be blown by a fan (108) and to be released, as shown by the arrows labelled as B or C or a combination thereof, preferably through the bottom outlets of the device (106).

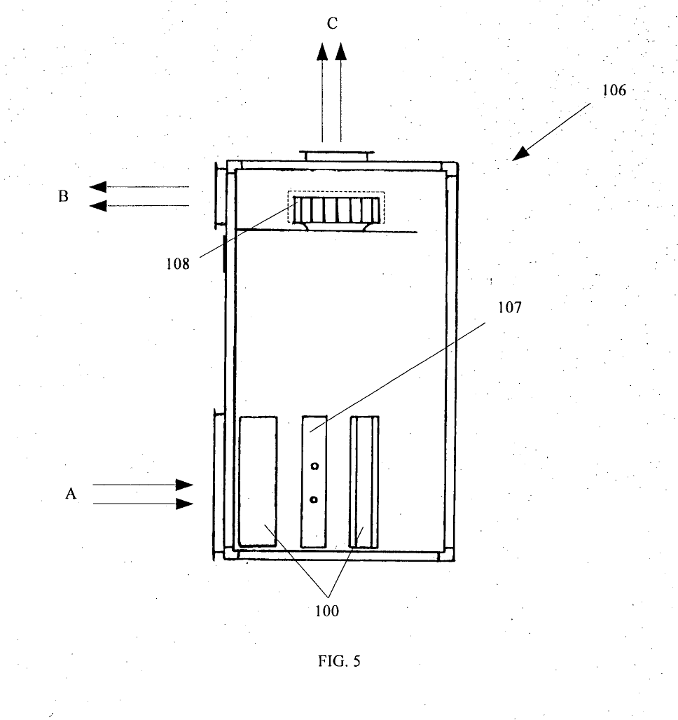

The preferred device (106) in FIG. 5. is another floor standing unit. In this device (106), air is also withdrawn as shown by the arrows labelled as A from an inlet and the air shall pass through at least one filter (100) which is positioned to be exposed to the ultraviolet light source (107). A fan (108) is used to blow the treated air through the outlets to around a point indicated as B or C or a combination thereof.

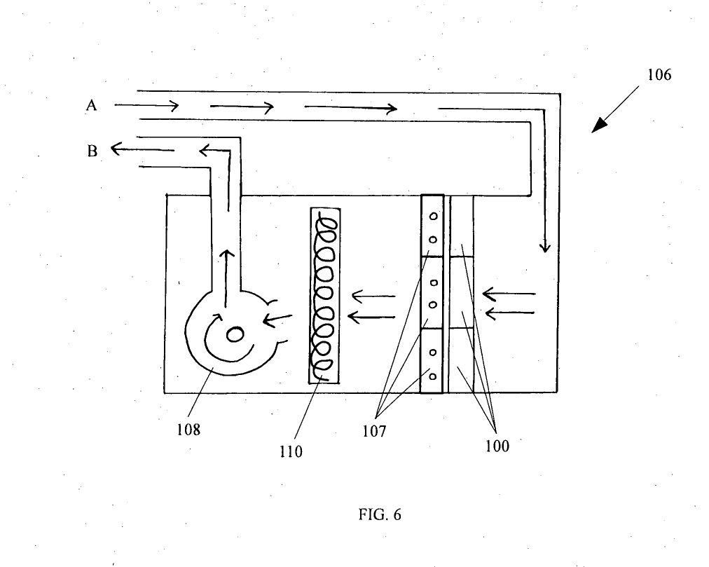

An air handling unit or fan coil unit is illustrated in FIG. 6 as the preferred device (106). The return air from around a point indicated as A is directed through a return air duct and into the device (106). The withdrawn air shall pass through at least one filter (100) which is exposed to at least one ultraviolet light source (107) and across a heating or cooling coil (110).

The heated or cooled air will be blown out of the device (106) by a fan (108) though a supply air duct to a point labelled as B. According to the present invention, the device (106) in FIG. 6 encloses the components preferably inside a plenum box. If there is absence of the plenum box, the components shall be preferably mounted on the return air side of the device (106).

Moreover, the present invention claims the ultraviolet light source (107) which radiates UVA and/or UVC but preferably at a wavelength ranges from 185 nm to 400 nm. The device (106) can be used in many places such as in the office, hotel guest room, airport, clean room and others.

As further disclosed in the present invention, the method of treating air comprising the steps of drawing air to a filter (100), exposing the filter (100) to an ultraviolet light source (107) to generate hydroxyl radicals and removing air contaminants by the hydroxyl radicals.

In the present invention, a repetitive oxidative reaction occurs at the titanium oxide surface on the ultraviolet photocatalytic layer (102) of a filter (100). In the redox reaction, the air which contains oxygen and water vapor is required for environmental purification using titanium oxide.

Photocatalysis is initiated when titanium dioxide exposes to the ultraviolet rays. This can be known as an optical solid surface or interface reaction. The titanium dioxide absorbs ultraviolet rays to generate electrons and positive-charged holes. Higher reaction effect is effected by a greater generation of the electrons and holes.

The reaction of photocatalysis is shown as below:
TiO₂+hv (ultraviolet rays)→e⁻+h⁺ (hole)

The generated holes have strong oxidization ability and by reacting with water present on the surface of the titanium dioxide, hydroxyl radicals can be generated to oxidize organic contaminant compounds. The hydroxyl radicals are generated through a reaction as shown below:
h⁺ (hole)+H₂O→.OH (hydroxyl radical)+H+

In the presence of oxygen, radicals of the intermediates of organic compounds and oxygen molecules induce a radical chain reaction and consume oxygen. The organic compounds shall be decomposed and eventually turn into carbon dioxide and water.

However, the generated electrons will produce superoxide anions by causing a reductive reaction with oxygen on the surface of the titanium dioxide, in which the reaction is shown as below:
e⁻+O₂→.O₂ (superoxide anion)

The superoxide anions can form oxides by adhering to the intermediates of the oxidation reaction or turn into hydrogen peroxide and then into water. Free oxygen radical (.O) is also generated in the air and directly affects the carbon-carbon bond of organic matter.

Since organic matter is usually more oxidizable than water, therefore the positive-charged holes are more likely to oxidize the organic compounds. The recombination rate of both carriers, which are the holes and electrons, shall decrease when the concentration of organic matter is higher.

The efficiency of the reaction is determined by the transfer of electrons to oxygen molecules at the reduction site under the condition where all of the spaces are filled.

1. A filter, comprising:
at least one antibacterial layer consisting of nano-silver particles; and
at least one ultraviolet photocatalytic layer.

2. A filter as claimed in claim 1, further comprising at least one electrostatic layer.

3. A filter as claimed in claim 1, wherein the antibacterial layer further includes zinc oxide, calcium oxide, magnesium oxide, silica or any combination thereof.

4. A filter as claimed in claim 1, wherein the antibacterial layer has a loading of nano-silver particles ranging from 0.1% to 5% by weight.

5. A filter as claimed in claim 1, wherein the nano-silver particles are ranging from 10 nm to 25 nm in size.

6. A filter as claimed in claim 1, wherein the ultraviolet photocatalytic layer consists of titanium oxide.

7. A filter as claimed in claim 6, wherein the ultraviolet photocatalytic layer has a loading of titanium oxide ranging from 0.1% to 21% by weight.

8. A filter as claimed in claim 7, wherein the filter is protected by a polymeric net, a metal casing or a combination thereof.

9. A device for treating air, comprising:
at least one ultraviolet light source; and
at least one filter comprising:
at least one antibacterial layer consisting of nano-silver particles; and
at least one ultraviolet photocatalytic layer;
wherein the filter is positioned to be exposed to the ultraviolet light source.

10. A device as claimed in claim 9, further comprising at least a fan, a coil or a combination thereof.

11. A device as claimed in claim 9, wherein the ultraviolet light source radiates at a wavelength ranging from 185 nm to 400 nm.

12. A device as claimed in claim 9, wherein the device has at least one surface coated with a loading of nano-silver particles ranging from 0.1% to 5% by weight and/or titanium oxide ranging from 0.1% to 21% by weight.

13. A method of treating air, comprising:
drawing air to a filter comprising:
at least one antibacterial layer consisting of nano-silver particles; and
at least one ultraviolet photocatalytic layer;
exposing the filter to an ultraviolet light source to generate hydroxyl radicals; and
removing air contaminants by the hydroxyl radicals.

14. A method as claimed in claim 13, wherein the ultraviolet light source radiates at a wavelength ranging from 185 nm to 400 nm.
